(19) United States
(12) Patent Application Publication
(10) Pub. No.: US 2014/0349283 A1
(43) Pub. Date: Nov. 27, 2014

(54) KIT USEFUL FOR DETECTING OF DONKEY MEAT PRESENT IN MEAT

(71) Applicants: Hasan Yetim, Ankara (TR); Fikrettin Sahin, Istanbul (TR); Zulal Kesmen, Kayseri (TR)

(72) Inventors: Hasan Yetim, Ankara (TR); Fikrettin Sahin, Istanbul (TR); Zulal Kesmen, Kayseri (TR)

(21) Appl. No.: 13/811,873

(22) PCT Filed: Jul. 23, 2011
(86) PCT No.: PCT/IB11/53292

§ 371 Date: Mar. 20, 2013

(30) Foreign Application Priority Data
Jul. 23, 2010 (TR) 2010/06092

(51) Int. Cl. C12Q 1/68

(52) U.S. Cl. CPC C12Q 1/6881; C12Q 2600/158
USPC 435/6.11

(57) ABSTRACT

The present invention relates to a kit comprising specific primer-probe set which is used for detecting donkey meat present in meat products by means of real-time PCR TaqMan probe technique. In the present invention contamination of donkey meat up to 0.1 picogram is enabled to be detected. The specific detection of donkey meat is made possible in raw and heat treated meat mixtures, by means of no cross-reaction until 40^{th} cycle with horse, pork, bovine, ovine, chicken and turkey species with the kit specific to the donkey species.

### FIELD OF THE INVENTION

The present invention relates to a kit comprising specific primer-probe set which is used for detection of donkey meat present in meat products by means of real-time PCR TaqMan probe technique.

### BACKGROUND OF THE INVENTION

Polymerase Chain reaction (PCR) briefly is an enzymatic amplification process of one or more target regions on the DNA through the short single-stranded oligonucleotide sequences (primers) complimentary to the 3′ ends flanking the segment of DNA to be amplified. In PCR applications, species identification can be performed by analyzing amplicons (PCR products) in further degree by using secondary methods after the specific gene or gene region belonging to any organism is amplified to obtain genetic material enough for the analysis. In recent years, real-time polymerase chain reaction (PCR) method which gives quantitative result is started to be used in order to detect the species in meat products. In real-time PCR method, amplification of the target gene is monitored by an increased fluorescence signal, increases proportionally to the amount of PCR product in a reaction.

In real-time PCR method, hydrolysis probe (TaqMan® probe) method is specifically preferred in studies performed to detect meat species as the intensity of the fluorescent signal emitted in parallel to the amount of the PCR product (amplicon) produced in each cycle. In this method fluorescent labeled oligonucleotid probes (a short single-stranded oligonucleotide molecule which is fluorescence marked) are used. It is designed to anneal to target sequence internally of the primers, during the annealing and extention phase of the PCR reaction. In its free, intact form no fluorescent emission can be measured, because fluorescent emission of the reporter dye is absorbed by quenching dye. However, upon annealing of the probe to one of the target strands, the probe will become degraded by the 5′-3′ exonuclease activity of the Taq polymerase. Consequently, the reporter and quencher dye become separated and the reporter dye emission is no longer transferred to the quenching dye, resulting in an increase of the reporter fluorescent emission. This process occurs in every cycle and does not interfere with the exponential accumulation of PCR products. The increase in fluorescence is measured cycle by cycle and directly correlates with the amount of the PCR product formed. The cycle number, at which the threshold value is exceeded is called “Ct (cycle threshold) value”, and it gives information about the starting quantity of the target DNA region.

PCR primers specific to target species produces a PCR product only in presence of the DNA to which they are specific under appropriate reaction conditions. The DNA fragment to be amplified is selected according to difference level which is shown for the detection of individual, population, species or the family for the purpose. The base sequences of the target DNA fragment to be used for species differentiation need to show maximum difference between species and minimum difference between individuals and populations within the species. Therefore the specificity of the oligonucleotide primer and probes to be used in amplification of the targeted gene region directly identifies the specificity of the method.

The first and only study about detecting donkey in meat products with real-time PCR TaqMan probe technique has been carried out by Chisholm et.al. They carried out a study on detection of horse and donkey species by using TaqMan probe method and they used mitochondrial cytochrome b (cytb) gene for the design of primer and probe [1]. But the method used in their study was not enough to differentiate species which are closely related after the 30^{th} cycle (Ct 30) and non-specific reactions occurred. Amplicon size is for horse species 69 by (base pair) and 119 by for donkey species. Horse specific primer and probe set showed non-specific reactions with bovine and donkey DNAs. And the primer and probe set specific to donkeys caused cross-reactions with horse DNA (Ct 30.77). In this application detection limit is found 25 ng for horse and 1 ng for the donkey.

Dooley et.al described a real-time PCR-based detection method in order to identify bovine, ovine, pork, turkey and chicken species tissues present in meat and meat products [2] by using specific primer and probe sets designed on the mitochondrial cytochrome b (cytb) The primer and probe sets specific to the pork showed cross-reactions with bovine, ovine, chicken and turkey. The Ct values detected for thesspecies are respectively 31.13, 37.08, 30.00, 34.64, and theoretically detection limit for pork is 0.02%.

Tanabe et.al designed primers and probes specific to pork, chicken, bovine, ovine and horses on the mitochondrial cytochrome b gene and detected each species in 100 fg/μl level with real-time PCR TaqMan technique [3].

Jonker et.al, also, developed a real-time PCR method for the identification of bovine, pork, horse, ovine, chicken and turkey species present in the processed meat products in 0.01% level by using species specific primer and probes sets. [4].

Frezza et.al could detected bovine and ovine species in 0.01-0.05 ng level by using 16S rRNA gene, and chicken and pork species in 0.5 ng level by using cytochrome b gene [5].

Koppel et.al developed multiplex real-time PCR method for detection bovine, pork, chicken and turkey species in 0.32-32 ng level with by using beta-actin and prolactin receptor genes [6].

International Patent document no WO2007119066, an application known in the state of the art, discloses a kit which enables the animal tissues in meats present in foods to be detected by using PCR technique.

### SUMMARY OF THE INVENTION

The objective of the present invention is to realize a kit comprising a specific primer probe set for detecting donkey meat in other meat products.

Another objective of the present invention is to realize a kit comprising a specific primer probe set which enables the donkey DNA up to 0.1 picogram in meat products to be detected. The other objective of the present invention is to realize a kit comprising a specific primer probe set which does not show any cross-reaction with DNA belonging to other animal species that can be present in reaction mixture until the 40^{th} cycle and thus only shows reaction with the donkey DNA and enables the donkey species to be detected specifically.

Yet another objective of the present invention is to realize a kit comprising a specific primer probe set which enables the donkey DNA in heat treated meat products to be detected.

### DETAILED DESCRIPTION OF THE INVENTION

“A kit for detecting donkey meat in meat products” realized to fulfill the objective of the present invention is illustrated in the accompanying figures wherein,

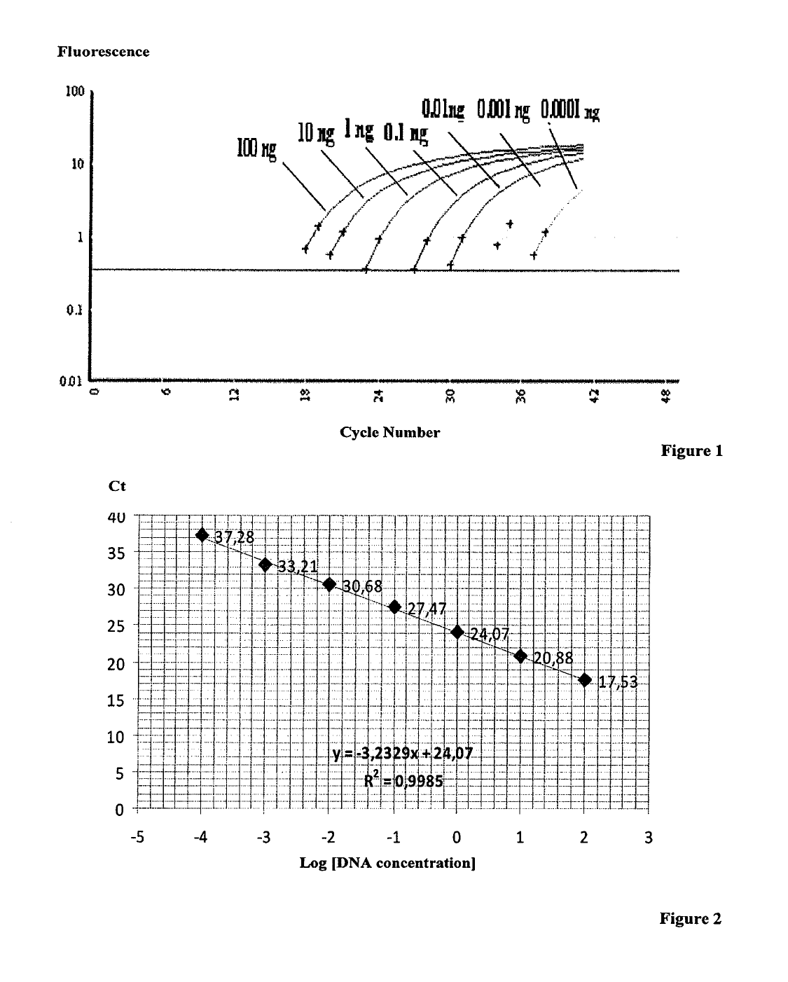

(FIG. 1) is the view of the fluorescence signals received in response to DNA dilution of donkey DNA between 0.0001 and 100 ng.

(FIG. 2) is the view of the linear relationships between Ct values detected in response to logarithmic concentrations of the donkey DNA.

The present invention comprises specific primer-probe set which is used in detecting donkey meat present in meat products by means of real-time PCR TaqMan probe technique. Specific primer-probe set is one of the components which are required for the detection of the donkey species with real-time PCR technique. Within the kit, there is a forward primer having length of 21 nucleotides designed specific to donkey species, a reverse primer having the length of 18 nucleotides specific to donkey species, and a TaqMan probe which is a hydrolysis probe having the length of 28 oligonucleotides which can be annealed specifically to the region amplified with the forward and reverse primers.

There is also nuclease enzyme free water and real time PCR reaction mixture within the kit. First DNA isolation should be done from the meat product in order to detect donkey species by using the kit. The purity of the isolated DNA needs to be high, it should not comprise PCR inhibitors and its 260/280nm ratio should be at least 1.7. The real-time reaction is performed in PCR tube of 200 μl and total volume of 50 μl. The reaction mixture is comprised of commercially available real-time PCR master mixture (25 μl) (in real-time PCR master mixture: HotStarTaq DNA Polymerase enzyme, PCR Buffer, dNTP mixture and 8 mM MgCl₂ is present), 0.8 μM forward and reverse primer, 0.2 μM TaqMan probe, 2 μM isolated DNA (its concentration is less than 500 ng) and 21.2 μl nuclease free water. In real time PCR device a filter appropriate for the wavelength at which the excitation and the emission of the fluorescence dye (reporter fluochrome) used in marking of the TaqMan probe is used . The temperature cycle is carried out as 15 seconds at 95° C., 1 minute at 60° C. for 40 cycles after 15 minutes of activation at 95° C.

The primer probe set present in the mentioned kit is comprised of a forward primer, a reverse primer and a dual-labeled oligonucleotid probe which are complementary target the region on mitochondrial DNA NADH dehydrogenase subunit 5 gene (ND5) (Chart 1). The primer-probe set includes a 21 nucleotides length forward primer, located between 11802-11823 nucleotides; a 20 nucleotides lenght reverse primer, located between 11867-11884 nucleotides; and a 28 nucleotides length dual-labeled oligonucleotid probe, located between 11827-11855 nucleotides on the ND5 gene (Acession number X97337) (Table 1). (Table 1).

In design of primer probe set, duplicate number and resistance against break downs with the effect of the heat of the mitochondrial DNA (MtDNA) is higher than the nuclear DNA (nDNA). For this reason the mitochondrial DNA is selected as it enables the detection of the donkey species which has mutation rate high enough to enable determination of closely related species to be separated even at low concentrations.

Primers and TaqMan probe set specific to the donkey species is designed on the mitochondrial ND5 (NADH dehydrogenase subunit 5) gene and it isadapted to the kit for detection of the donkey. The nucleotide length and mutation degrees of the ND5 gene issufficient to design species specific primer-probe. Therefore in real-time PCR method realized by using primer and probe set for detection of donkey meat the reaction is continued even until the 40^{th} cycle cross-reaction does not occur with other animal species. And this increases the sensitivity of the developed method.

The nucleotid sequences of the primers designed specific to donkey species and, length and the genomic localizations of the amplification products are given in Table 1.

TABLE 1
Nucleotidsequences of primer and probe set
used for detection of the donkey species
and genomic localization information.

| | | Base | Genomic | Ampli- |
|---|---|---|---|---|
| | Oligonu- | sequence | local- | con |
| Species | cleotide | (5′-3′) | ization | length |
| Donkey | Forward | 5′-TGCTAGCCTCA | ND5 | 83 bp** |
| | Primer | TTATCAGTAT-3′ | (11802- | |
| | | | 11823) | |
| | Reverse | 5′-GTGATGAGGAT | ND5 | |
| | Primer | ACGTGCT-3′ | (11867- | |
| | | | 11884) | |
| | TaqMan | 5′***TCTACCAAT | ND5 | |
| | Probe | CATATCATCAATCC | (11827- | |
| | | TCAAC-3′**** | 11855) | |

*ND5: NADH dehydrogenase subunit 5 gene
**bp: base pair
***Reporter dye
****Quencher dye

And the comparison of oligonucleotide sequences of primer and probe set with oligonucleotide sequences of target gene region of the widely consumed other animal species is given at Chart 1.

CHART 1
The comparison of target DNA of donkey with other animal species

| Donkey | 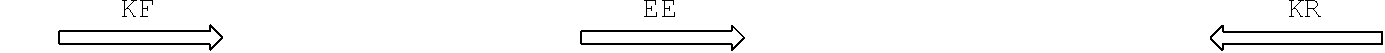 |
|---|---|
| Donkey | 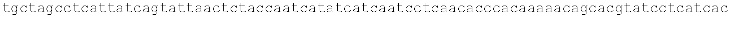 |
| Horse | .a....tt...c...t....c....c..c..........t.......t....t...t...............tc..g.....t |
| Pork | cat..a..a..c..atta.tc....ca.c.....t...at...c.a.tca....t.t......ct.a.ctt..c...a.ct.. |
| Bovine | ca....tta.t...ctct..c.....ac...c..t...at.at..g.t.t......t.....ccttc..ac..c..a.tct.. |
| Ovine | ca....t.a.cc..attc..c....ca....c...gc.g..atc.att.t........t...tt..c..at.....a.tct.t |
| Chicken | cc..ca.aa.....a..c..c.c..c..c.tt.c.cctattatcc.t.c.cc.ct..ta..tcta.aa.ac.cc..catatc. |
| Turkey | cc..ca.tct.c..a.ca..c.t.t...t.tc.c.cc.attattt.atcacc.tta.ta..cct..aa.at.cc.....atta |

When the nucleotide sequences of primer and probe specific to donkey species is compared with the nucleotide sequences of the other widely consumed animal species, it is observed that forward primer is different with 6 bp, probe is different with 5 bp and the reverse primer is different with 4 bp from the horse species to which is the closest species genetically (Chart 1). This enables the specific detection of donkey species.

The donkey meat can be detected in meat products like salami, sausage, meatball, canned meat etc. PCR product can only be obtained in presence of donkey DNA by using primer-probe set specific to donkey species in meat products.

In Table 2 the CT values of the designed primers and probe detected with the different devices are given. It is seen that the detection of the donkey species is possible up to 0.0001 ng with two different devices with primer-probe set designed.

TABLE 2
Sensitivity test results of primer-
probe specific to the donkey species

| | |
|---|---|
| | Ct values measured with |
| oncentration | two different devices |

| of donkey | (Line) Gene II | IQ5 |
|---|---|---|
| DNA (ng) | (Bioer) | (BioRad) |
| 100 | 17.53 | 16.79 |
| 10 | 20.88 | 19.29 |
| 1 | 24.05 | 23.09 |
| 0.1 | 27.47 | 27.34 |
| 0.01 | 30.68 | 30.85 |
| 0.001 | 33.21 | 35.43 |
| 0.0001 | 37.28 | 38.59 |

Specificity test results of primer-probe specific to the donkey species are given at Table 3. According to this, it is observed that each one of the primer and probes specific to donkey species does not show cross-reaction with other tested animal species until the 40^{th} cycle.

TABLE 3
Specificity test results of primer-
probe specific to the donkey species

Ct values measured with
two different devices

| Species | (Line) Gene II | IQ5 |
|---|---|---|
| (100 ng DNA) | (Bioer) | (BioRad) |
| Donkey | 17.53 | 16.79 |
| Horse | ND | ND |
| Pork | ND | ND |
| Bovine | ND | ND |
| Ovine | ND | ND |
| Chicken | ND | ND |
| Turkey | ND | ND |

ND: Not detected

In real time PCR a standard curve obtained by plotting Ct values (cycle number which the fluorescence signal is first detected) against logaritma concentrations of serial ten-fold dilutions of the target nucleic acid is a very useful tool for determining the qualities of an assay. In FIG. 2 a constructed standard curve by using Ct values obtained from a serial 10 fold dilution of donkey DNA (ranged from 0.0001 to 100 ng DNA) is given. The assay efficiency, which is a function of the slope, is primarily an indication of how well the PCR reaction has proceeded. When the slope of the standard curve is 3.33, the efficiency of the real-time PCR is accepted as 100%. The slope of the standard curve plotted with the primer-probe set designed specific to the donkey species is 3.23, which is found very close to the 3.33 value. It is determined that the correlation between the DNA concentrations and the Ct values is 0.999 (FIG. 2). The presence of a linear relationship between the ct values and DNA concentrations enables the donkey species to be detected quantitatively with high accuracy between 0.0001-100 ng DNA concentrations with the designed primer-probe set.

Ct values detected for raw and cooked meatballs are seen in Table 4. With the aim of testing the effect of the heat treatment applied in production of meat products to the accuracy of the invented kit, meat mixtures are prepared by adding donkey meat in different amounts (0.0001, 0.001, 0.01, 0.1, 1, 10 and 100 ng) to beef. Two different heat treatments are applied to the mentioned meat mixtures either at 200° C. for 30 min. or at 120° C. under the pressure of 15 psi (autoclave) for 30 min. It is determined that TaqMan probe method with primer-probe set specific to donkey gives successful results and the detection limit is 0.001 ng for the samples heat treated at 200° C. for 30 min and 0.01 ng for the samples heat treated at 120° C. under 15 psi for 30 min. Therefore it is found that heat treatment applied on the meat products has no negative effect on the sensitivity of the method for the detection of meats from donkey species with the mentioned method (Table 4).

TABLE 4
Detected Ct values for raw and cooked meatballs

| Target | Rate of the | | Ct values of | Ct values of |
|---|---|---|---|---|
| species | donkey meat in | Ct values | samples heat | samples heat |
| being | binary mixture | of raw | treated at | treated at |
| analysed | (%) | samples | 120° C. | 200° C. |
| DONKEY | 10 | 21.23 | 24.24 | 20.76 |
| | 1 | 24.1 | 27.52 | 24.57 |
| | 0.1 | 27.78 | 29.75 | 27.12 |
| | 0.01 | 30.1 | 34.78 | 30.43 |
| | 0.001 | 33.53 | ND | 34.78 |
| | 0.0001 | 36.68 | ND | ND |

ND: Not detected

The invented kit is appropriate for the detection of donkey meat in beef up to the level of 0.0001% Therefore it is appropriate for detection of adulteration commonly performed by mixing beef with donkey meat.

The invented kit is appropriate for detection of donkey meat in raw meat and meat heat treated at 120° C. under the pressure of 15 psi, and at oven temperature of 200° C. for 30min. therefore, it can be used in cooked meat products or canned meat.

### REFERENCES

1. Chisholm, J., Conyers, C., Booth, C., Lawley, W., Hird, H.; “The detection of horse and donkey using real-time PCR”, Meat Science 70 (2005) 727-732.
2. John J. Dooley, Kelly E. Paine, Stephen D. Garrett, Helen M. Brown; “Detection of meat species using TaqMan real-time PCR assays”, Meat Science 68 (2004) 431-438.
3. Tanabe, S., Hase, M., Yana, T., Sato, M., Fujimura, T., Akiyama, H.; “A Real-Time Quantitative PCR Detection Method for Pork, Chicken, Beef, Mutton, and Horseflesh in Foods”. Bioscience, Biotechnology, and Biochemistry Vol. 71 (2007), No. 12 pp.3131-3135.
4. Jonker, K. M., Tilburg, J. J. H. C., HäGele, G. H., De Boer, E.; “Species identification in meat products using real-time PCR. Food Additives and Contaminants,” (2008), 25(5): 527-533.
5. Frezza, D., Giambra, V., Chegdani, F., Fontana, C., Maccabiani, G., Losio, N., et al.; “Standard and Light-Cycler PCR methods for animal DNA species detection in animal feedstuffs .” Innovative Food Science and Emerging Technologies 9 (2008), 18-23.
6. Koppel, R., Ruf, J., Zimmerli, F., Breitenmoser, A.; “Multiplex real-time PCR for the detection and quantification of DNA from beef, pork, chicken and turkey.” Eur Food Res Technol (2008), 227:1199-1203

1-6. (canceled)

7. A kit for detecting donkey meat in meat products comprising: a forward primer, a reverse primer; and a probe which are complementary to the region on mitochondrial NADH dehydrogenase subunit 5 (ND5) gene; and a primer-probe set in detection of donkey meat present in the meat products with real-time PCR TaqMan probe technique.

8. The kit according to claim 7, further comprising: a 21 nucleotides length forward primer, located between 11802-11823 nucleotides; a 20 nucleotides lenght reverse primer located between 11867⁻11884 nucleotides; and a 28 nucleotides length dual-labeled oligonucleotid probe located between 11827-11855 nucleotides on the ND5 gene of donkey (Genbank Accession number X97337).

9. The kit according to claim claim 7 wherein the primer-probe set which shows reaction only with the DNA of the donkey species until 40th cycle for detection of donkey meat present in meat products with real-time PCR TaqMan probe technique.

10. The kit according to claim 8 wherein the primer-probe set which shows reaction only with the DNA of the donkey species until 40th cycle for detection of donkey meat present in meat products with real-time PCR TaqMan probe technique.

11. The kit according to claim 7 wherein the primer-probe set can be applied to meat products such as salami, sausage, meatball and calmed meat wherein the donkey meat is detected.

12. The kit according to claim 8 wherein the primer-probe set can be applied to meat products such as salami, sausage, meatball and calmed meat wherein the donkey meat is detected.

13. The kit according to claim 9 wherein the primer-probe set can be applied to meat products such as salami, sausage, meatball and calmed meat wherein the donkey meat is detected.

14. The kit according to claim 10 wherein the primer-probe set can be applied to meat products such as salami, sausage, meatball and calmed meat wherein the donkey meat is detected.

15. The kit according to claims 7 wherein the primer-probe set can be applied to raw meat and meat product heat treated at 120° C. and under the pressure of 15 psi.

16. The kit according to claim 8 wherein the primer-probe set can be applied to raw meat and meat product heat treated at 120° C. and under the pressure of 15 psi.

17. The kit according to claim 9 wherein the primer-probe set can be applied to raw meat and meat product heat treated at 120° C. and under the pressure of 15 psi.

18. The kit according to claim 10 wherein the primer-probe set can be applied to raw meat and meat product heat treated at 120° C. and under the pressure of 15 psi.

19. The kit according to claim 11 wherein the primer-probe set can be applied to raw meat and meat product heat treated at 120° C. and under the pressure of 15 psi.

20. The kit according to claim 12 wherein the primer-probe set can be applied to raw meat and meat product heat treated at 120° C. and under the pressure of 15 psi.

21. The kit according to claim 13 wherein the primer-probe set can be applied to raw meat and meat product heat treated at 120° C. and under the pressure of 15 psi.

22. The kit according to claim 14 wherein the primer-probe set can be applied to raw meat and meat product heat treated at 120° C. and under the pressure of 15 psi.

23. The kit according to claim 1 wherein the primer-probe set can be applied to a heat treated meat product at 200° C. for 30min.
